(19) United States
(12) Patent Application Publication
(10) Pub. No.: US 2026/0235598 A1
(43) Pub. Date: Aug. 13, 2026

(54) SILVER NANOPARTICLE BASED ELECTROCHEMICAL BIOSENSOR USING SCREEN PRINTED CARBON ELECTRODE

(71) Applicant: UNIVERSITY OF UTAH RESEARCH FOUNDATION, Salt Lake City, UT (US)

(72) Inventors: Dhruv PATEL, Salt Lake City, UT (US); Shruti HEGDE, Salt Lake City, UT (US); Himanshu J. SANT, Salt Lake City, UT (US)

(21) Appl. No.: 19/470,692

(22) PCT Filed: Mar. 29, 2024
(86) PCT No.: PCT/US2024/022351

§ 371 Date: Sep. 29, 2025

Related U.S. Application Data
(60) Provisional application No. 63/493,465, filed on Mar. 31, 2023.

(51) Int. Cl. G01N 33/543; G01N 27/327

(52) U.S. Cl. CPC G01N 33/5438; G01N 27/3272; G01N 27/3278

(57) ABSTRACT

Disclosed is an electrochemical biosensor using silver nanoparticles (AgNPs) and screen printed carbon electrodes (SPCEs) that may be used to detect Shiga toxin. The AgNPs are modified with antibodies with specificity to a desired antigen (e.g., Shiga toxin-1). The reference electrode of the SPCEs may be modified with bleach, such that the reference electrode does not significantly contribute to a background signal while measuring the concentration of AgNPs. Related methods can isolate modified AgNP bound antigen molecules, dissolve the modified AgNPs in solution, and detect a concentration of AgNPs using an electrochemical assay that employs the modified SPCE. This detected concentration of AgNPs can subsequently be used to determine the concentration of the target antigen, wherein the concentration of the AgNPs directly correlates to the concentration of the target antigen.

### CROSS-REFERENCE TO RELATED APPLICATIONS

This application claims the benefit of and priority to U.S. Provisional Patent Application Ser. No. 63/493,465 filed on Mar. 31, 2023, and entitled “SILVER NANOPARTICLE BASED ELECTROCHEMICAL BIOSENSOR USING SCREEN PRINTED CARBON ELECTRODE,” which application is expressly incorporated herein by reference in its entirety.

### STATEMENT REGARDING FEDERALLY SPONSORED RESEARCH OR DEVELOPMENT

N/A.

### BACKGROUND

The economic and health cost of Shiga toxin poisoning in humans is significant, and it is becoming an ever-increasing challenge. For example, a 2017 Centers for Disease Control and Prevention report suggests that incidences of Shiga toxin-producing Escherichia coli (STEC) infection per 100,000 population reported to Laboratory-based Enteric Disease Surveillance increased by 132% from 1997 to 2017. The estimated economic cost of STEC infections is greater than $400 million annually. Shiga toxins damage endothelial cells in the kidney and brain, causing renal failure and neurological complications. Shiga toxin has more hospitalizations per infection than other foodborne illnesses caused by Salmonella or Campylobacter.

The need for a reliable and sensitive detection method for Shiga toxin stems from the very small infectious dosage to humans and animals. A microgram quantity of Shiga toxin can be lethal and can cause kidney failure, neurological complications, and/or necrosis of tissues. It is estimated that 63.7μg of Shiga toxin-1 is a lethal amount for a 45.5 kg human. Similarly, the calculated LD₅₀ in mice is from 20 μg/kg to 450 μg/kg, making Shiga toxin very dangerous.

Additionally, there is no practical way to treat Shiga toxin poisoning. Some antibodies induce the replication of phases, so antibody treatment is not recommended for Shiga toxin poisoning. Due to the lack of available treatments, detecting Shiga toxin before its consumption or spread as a preventive measure is necessary. Hence, rapid, point-of-use, low-cost, and reliable detection of Shiga toxin-1 would be incredibly helpful as a preventive tool.

### BRIEF SUMMARY

Disclosed herein are various embodiments and methods relating to electrochemical biosensors using silver nanoparticles (AgNPs) and screen-printed carbon electrodes (SPCEs). The described electrochemical biosensors described herein are useful for antibody-based detection of antigens such as Shiga toxin. Detection of other toxins or other analytes may of course also be possible.

An exemplary embodiment of an electrochemical biosensor comprises silver nanoparticles (AgNPs) with antibodies attached to the AgNPs. The electrochemical biosensor further comprises a screen printed carbon electrode (SPCE) or other electrode, wherein the electrode comprises a reference electrode. The reference electrode comprises an outer surface. The outer surface of the reference electrode may comprise a silver coating wherein the outer surface has been passivated.

In any of the described embodiments, the outer surface of the reference electrode can be modified by treatment with bleach (e.g., hypochlorite). Treatment of the outer surface with bleach produces AgCl on the outer surface. Furthermore, treatment of the outer surface with bleach removes Ag⁺ ions from the outer surface. In some embodiments, after treatment with bleach, the outer surface is substantially free of free Ag⁺ ions. Such embodiments (i.e., embodiments wherein the reference electrode is treated with bleach) allow SPCEs to reliably and sensitively detect silver nanoparticles (AgNPs).

In any of the described embodiments, the electrochemical biosensor may further comprise magnetic nanoparticles (MNPs) wherein the MNPs include antibodies attached thereto.

In any of the described embodiments, the antibodies attached to the AgNPs may comprise Anti-Shiga Toxin, for example, Camelid Antibody, VHH (#761L, List Labs, Campbell, CA, USA) herein described as Vh-antibodies. In any of the described embodiments, the antibodies attached to the MNPs may comprise Anti-Shiga Toxin 1, e.g., (Rabbit), lgG (#761L, List Labs, Campbell, CA, USA) herein referred to as R-antibodies.

In any of the described embodiments, an electrochemical biosensor may comprise AgNPs including antibodies attached thereto. The electrochemical biosensor may further comprise an SPCE, wherein the SPCE comprises a reference electrode. The reference electrode may comprise an outer surface, wherein the outer surface may comprise a platinum coating.

A related method involves providing an electrochemical biosensor. The electrochemical biosensor comprises silver nanoparticles with antibodies attached to the silver nanoparticles and a carbon electrode (e.g., a SPCE) comprising a reference electrode. The reference electrode may comprise an outer surface wherein the outer surface comprises a silver coating and wherein the outer surface has been passivated (e.g., with bleach). The method further involves attaching the silver antibodies to an analyte. The method further involves using the reference electrode to detect the silver nanoparticles and determine a detected concentration of the analyte.

In any of the described embodiments, the analyte to be measured by the electrochemical detection method may be Shiga toxin. Using the method described herein, Shiga toxin may be reliably detected at a threshold concentration of about 2 ng/ml. Even lower threshold concentrations may be possible, e.g., 1 ng/mL, for example, by optimizing the presently described embodiments.

In any of the described embodiments, the electrochemical detection method may be performed in less than about 3 hours.

Such embodiments provide advantages of low cost (e.g., as compared to use of gold nanoparticles, which are significantly more expensive), as well as a significantly more linear calibration curve, as compared to alternative techniques.

In addition to the described use of carbon electrodes, other electrode materials are also possible. For example, a platinum-based reusable sensor may alternatively be used.

In an embodiment, the employed AgNPs may have a size (e.g., average diameter) of less than about 500 nm, less than about 400 nm, less than about 300 nm, less than about 200 nm, less than about 100 nm, less than about 80 nm, less than about 50 nm, such as from about 1 nm to about 50 nm, from about 5 nm to about 40 nm, or from about 10 nm to about 30 nm (e.g., about 20 nm).

In an embodiment, the employed magnetic nanoparticles may have a size (e.g., average diameter) that is significantly greater than that of the AgNPs. By way of example, the magnetic nanoparticles may have a size of less than about 10 μm, less than about 5 μm, less than about 3 μm, less than about 2 μm, greater than about 100 nm, greater than about 200 nm, greater than about 300 nm, greater than 400 nm, or greater than 500 nm, such as from about 300 nm to about 5 μm, from about 400 nm to about 3 μm, or from about 500 nm to about 2 μm (e.g., about 1 μm).

Features from any of the disclosed embodiments may be used in combination with one another, without limitation. This summary is provided to introduce a selection of concepts in a simplified form that are further described below in the detailed description. This summary is not necessarily intended to identify key or essential features of the claimed subject matter, nor is it intended to be used as an indication of the scope of the claimed subject matter.

Additional features and advantages will be set forth in part in the description that follows, and in part will be obvious from the description, or may be learned by practice of the embodiments disclosed herein. It is to be understood that both the foregoing brief summary and the following detailed description are exemplary and explanatory only and are not restrictive of the embodiments disclosed herein or as claimed.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings.

FIG. 1 schematically illustrates the chemical relationship between an exemplary electrochemical biosensor and an antigen.

FIG. 2 illustrates an exemplary embodiment of a screen printed carbon electrode (SPCE).

FIG. 3 schematically illustrates an exemplary electrochemical detection method.

FIG. 4 illustrates a bar graph displaying antibody binding efficiency.

FIG. 5 illustrates a CV curve of the modified SPCE.

FIGS. 6 and 7 illustrate Shiga toxin detection results from an exemplary electrochemical detection method using silver nanoparticles (AgNPs).

FIG. 8 illustrates Shiga toxin detection results from an exemplary electrochemical detection method using gold nanoparticles (AuNPs).

### DETAILED DESCRIPTION OF THE PREFERRED EMBODIMENTS

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “nanoparticle” includes one, two or more nanoparticles.

Unless otherwise stated, all percentages, ratios, parts, and amounts used and described herein are by weight.

Numbers, percentages, ratios, or other values stated herein may include that value, and also other values that are about or approximately the stated value, as would be appreciated by one of ordinary skill in the art. As such, all values herein are understood to be modified by the term “about” or its synonyms such as “approximately” or “substantially.” Such values thus include an amount or state close to the stated amount or state that still performs a desired function or achieves a desired result. A stated value should therefore be interpreted broadly enough to encompass values that are at least close enough to the stated value to perform a desired function or achieve a desired result, and/or values that round to the stated value. The stated values include at least the variation to be expected in a typical manufacturing or other process, and may include values that are within 10%, within 5%, within 1%, etc. of a stated value.

Some ranges may be disclosed herein. Additional ranges may be defined between any values disclosed herein as being exemplary of a particular parameter. All such ranges are contemplated and within the scope of the present disclosure.

As used herein, the term “between” is inclusive of any endpoints noted relative to a described range. For example, “between 2 and 10” includes both 2 and 10.

In the application, effective amounts are generally those amounts listed as the ranges or levels of ingredients in the descriptions, which follow hereto. Unless otherwise stated, amounts listed in percentage (“%'s”) are in weight percent (based on 100% active) of any composition.

The phrase ‘free of’ or similar phrases if used herein means that the composition or article comprises 0% of the stated component, that is, the component has not been intentionally added. However, it will be appreciated that such components may incidentally form thereafter, under some circumstances, or such component may be incidentally present, e.g., as an incidental contaminant.

The phrase ‘substantially free of’ or similar phrases as used herein means that the composition or article preferably comprises 0% of the stated component, although it will be appreciated that very small concentrations may possibly be present, e.g., through incidental formation, contamination, or even by intentional addition. Such components may be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the compositions or articles described herein may be free or substantially free from any specific components not mentioned within this specification.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Although the systems and methods described herein are described in the context of detecting Shiga toxin-1, other toxins, antigens and the like may be detected using the disclosed systems and methods without departing from the spirit and scope of the invention.

### II. General Overview of Example Embodiments

Disclosed embodiments utilize silver nanoparticles (AgNPs) with antibodies attached thereto to isolate a desired antigen. Furthermore, disclosed embodiments may utilize carbon electrodes, such as screen printed carbon electrodes (SPCEs) to measure silver nanoparticles and determine a measured concentration of the desired antigen. The electrochemical biosensors and related methods enjoy high specificity, low economic costs, and are easily translated into point-of-use systems. Furthermore, the electrochemical biosensors utilize low cost, and readily available SPCEs for rapid, sensitive, and reliable detection of a desired antigen such as Shiga toxin-1.

As described above, food, wastewater, and other substances contaminated with Shiga toxin may infect humans and/or animals, inflicting significant health and economic costs. Hence, there is a need for rapid, point-of-use, low cost, and reliable methods to detect Shiga toxin contaminations. Methods well known in the art, such as enzyme immunoassay (EIA) and polymerase chain reaction (PCR) approaches are reliable, but require expensive instruments and reagents, making these methods difficult to translate into low cost, point-of-use systems.

As a result, alternative systems for Shiga toxin detection are being studied. For example, biosensors utilizing gold nanoparticles (AuNPs) have been proposed to detect Shiga toxin contaminations. However, such systems have higher detection limits and are less reliable than EIA or PCR approaches.

The exemplary biosensors utilizing AgNPs described enjoy significant advantages over other Shiga toxin detection approaches known in the art. For example, the advantages of using AgNP-based electrochemical biosensors over AuNP-based electrochemical biosensors are described as follows. First, for the yes/no test (a test that only indicates results as positive or negative instead of concentration), the test merely requires a fixed voltage battery with an acid capable of dissolving silver (e.g., nitric acid (HNO₃)) for point-of-use detection in remote areas. Other acids that may dissolve silver include perchloric acid and/or sulfuric acid (e.g., particularly when heated) Second, AgNPs are 90 times cheaper than AuNPs, significantly reducing the economic cost of using such systems. The electrochemical dissolution of nanoparticles also determines their electrochemical efficiency, and AgNPs are easier to dissolve and modify than AuNPs, according AgNP based electrochemical biosensors enhanced reliability and lower detection limits when compared to AuNP based electrochemical biosensors. Comparative results are presented and described hereinbelow. Furthermore, the AgNP based electrochemical biosensors described herein can employ low-cost and readily available SPCEs to detect Shiga toxin.

Embodiments as described herein utilize modified AgNPs (i.e., AgNPs with antibodies attached thereto) to bind and detect Shiga toxin-1.

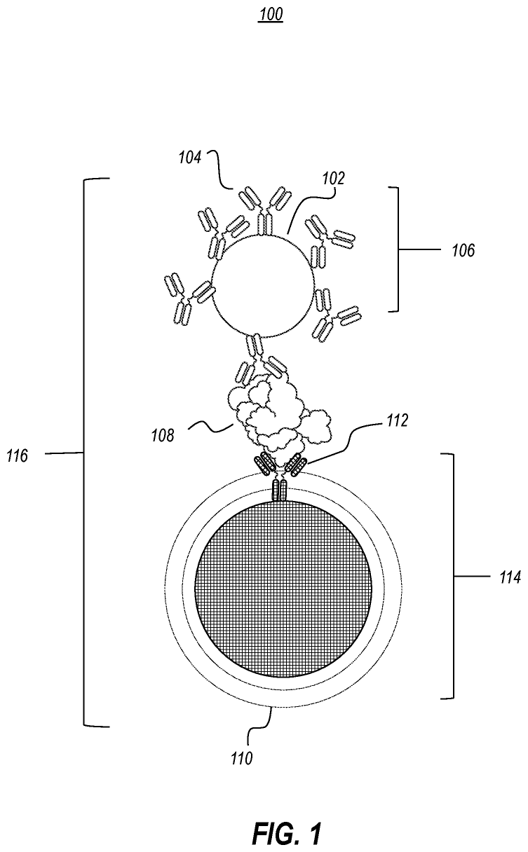

FIG. 1 illustrates an electrochemical biosensor 100. The electrochemical biosensor 100 comprises AgNPs 102 wherein detection antibodies 104 are attached thereto. The AgNPs 102 and detection antibodies 104 form modified AgNPs 106. Modified AgNPs 106 may bind antigen 108 via detection antibodies 104, wherein detection antibodies 104 comprise antibodies with specificity to antigen 108. Exemplary methods of modifying AgNPs with antibodies are described herein.

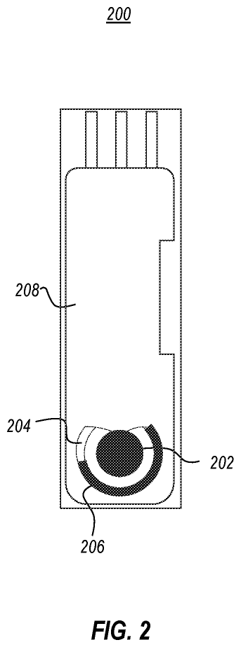

Electrochemical biosensor 100 is further used with a SPCE. Attention is now turned to FIG. 2, which illustrates SPCE 200. SPCE 200 comprises working electrode 202, reference electrode 204, and counter electrode 206. Reference electrode 204 comprises an outer surface. In some embodiments SPCE 200 may comprise insulation 208.

The outer surface of reference electrode 204 may comprise a silver coating. In some embodiments, the silver coating disposed on the outer surface of reference electrode 204 may be modified with bleach (e.g., hypochlorite bleach). Modification with bleach may improve the performance of SPCEs when used to detect silver nanoparticles. SPCEs as described herein may employ the addition of HNO₃ in a buffer to detect nanoparticles such as AgNPs. However, HNO₃ reacts with the silver coating disposed on the outer surface of the reference electrode, dissolving the silver coating. Dissolved silver from the outer surface of reference electrode 204 would contribute to a background signal while measuring AgNPs, thereby generating interference and creating a variable detection signal. Modification with bleach ameliorates this problem because bleach reacts with the silver coating disposed on the outer surface of reference electrode 204, transforming exposed silver into AgCl. Due to the extremely low Ksp (solubility constant in water) of AgCl, silver from the modified reference electrode does not react with and/or dissolve into the HNO₃ buffer to any significant degree. An exemplary experiment directed to the characterization of SPCEs modified with bleach is described herein. Furthermore, an exemplary experiment directed to validating the modified SPCEs as a viable electrode system for detecting AgNPs is described herein.

In some embodiments, the silver coating disposed on the outer surface of reference electrode 204 may be replaced, in full or in part, by a platinum coating. In such embodiments, passivation (e.g., with bleach) may not be needed.

Referring back to FIG. 1, electrochemical biosensor 100 may further comprise magnetic nanoparticles (MNPs) 110 with capture antibodies 112 attached thereto. MNPs 110 and capture antibodies 112 form modified MNPs 114. Modified MNPs 114 may bind antigen 108 via capture antibodies 112, wherein capture antibodies 112 comprise antibodies with specificity to antigen 108.

In some embodiments detection antibodies 104 and capture antibodies 112 may comprise the same antibodies. For example, detection antibodies 104 and capture antibodies 112 may both comprise an anti-Shiga toxin, such as Anti-Shiga Toxin 1 (Rabbit), lgG (#761L, List Labs, Campbell, CA, USA) (herein described as R-antibodies). In some embodiments, detection antibodies 104 and capture antibodies 112 may comprise different antibodies. For example, in an exemplary embodiment, detection antibodies 104 may comprise Camelid Antibody, VHH (#761L, List Labs, Campbell, CA, USA) (herein described as Vh-antibodies) while capture antibodies 112 may comprise R-antibodies. An exemplary experiment to determine effective antibodies for binding Shiga toxin-1 is described herein. Various other detection and/or capture antibodies could alternatively be used (e.g., using methods as described, to identify suitable candidates). As depicted in FIG. 1, detection antibodies 104 and capture antibodies 112 bind antigen 108 simultaneously, creating antigen detection complex 116.

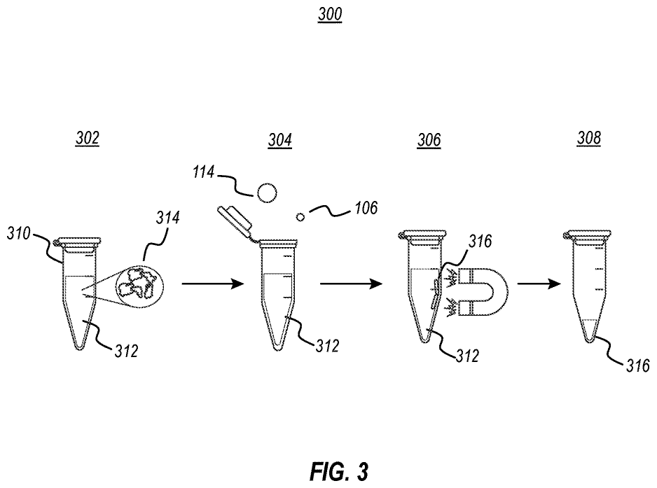

The biosensors described herein, such as the exemplary biosensor described with reference to FIGS. 1 and 2 may be used to detect a desired antigen such as Shiga toxin-1. FIG. 3 depicts a schematic of a method 300 that utilizes the electrochemical biosensors described herein to bind a desired antigen. Method 300 comprises step 302, wherein step 302 involves providing a vial or other container 310 containing solution 312, wherein solution 312 may contain a detectable quantity of antigen 314. Antigen 314 may be represented as antigen 108 in FIG. 1. Solution 312 may be a sample prepared for the purpose of testing a substance for a desired antigen. By way of illustration, solution 312 may comprise a wastewater sample and/or a dissolved food sample. Method 300 further comprises step 304, wherein step 304 involves mixing modified AgNPs 106 and modified MNPs 114 into solution 312 such that modified AgNPs 106 and modified MNPs 114 bind antigen 314, forming antigen detection complex 116 (see FIG. 1). Method 300 further comprises step 306, wherein step 306 involves using an immunomagnetic separation (IMS) protocol to separate antigen detection complex 116 from solution 312, forming pellet(s) 316. After step 306, solution 312 is preferably free or substantially free of antigen 314, wherein all, or substantially all, of antigen 314 is captured in pellet 316. Method 300 further comprises step 308, wherein step 308 involves condensing pellet(s) 316 and removing solution 312.

Pellet(s) 316 can be resuspended and analyzed using an SPCE 200 (see FIG. 2). In some embodiments, pellet(s) 316 is resuspended in HNO₃ in buffer, wherein HNO₃ dissolves modified AgNPs 106 into the buffer. The AgNP concentration may thus be measured using an SPCE 200. Because modified AgNPs 106 are highly soluble in such a buffer (100% dissolution and stripping within about 1 s), 1 AgNP would create a signal of 1 pA, which modern instruments can measure. Hence, the detection of one antigen 314 molecule connected to a modified AgNP 106 in an ideal system with currently available technology would theoretically be possible. Although various factors such as antibody binding efficiency prevent actual systems from being ideal, the electrochemical biosensor and related methods described herein enjoy very sensitive detection limits, within the same order of sensitivity as more costly approaches such as EIA or PCR. Specifically, the electrochemical biosensors described herein achieve a lowest detectable concentration of 2 ng/ml for Shiga toxin-1 in less than 3 hours. An exemplary assay and related results are presented and described herein.

### III. Examples and Results

AgNPs were modified (e.g., with reference to FIG. 1 and the corresponding description) using exemplary procedure 1:960 μL of AgNPs (60 nm) were mixed with 40 μL of 2 mM borate buffer to adjust the pH to 8.5. After that, the AgNPs were modified with 10 μL of (multi-monolayer) MML solution. MML solution contains 1.0 mM of disodium phosphate (DSP) and 10 mM of methiopropane (MPA) in acetonitrile. The solution was rotated for 2 hours in rotating plates. Antibodies were attached to MML-modified AgNPs by adding 5.0 μg of the desired antibodies and mixed on a rotating plate for 3 more hours. Next, the solution was added to 100 μL of 10% (v/v) bovine serum albumin (BSA) in 2 mM borate buffer and rotated for 6-8 hours. Finally, AgNPs were washed with centrifugation at 2500 g for 12 minutes, removing the supernatant and resuspending the AgNP pellet into 1 mL of 1% (v/v) BSA in 2 mM borate buffer.

MNPs were modified (e.g., with reference to FIG. 1 and the corresponding description) using exemplary procedure 2: Magnetic beads were modified with antibodies following instructions from the Dynabeads_myone manual from Thermo Fischer. In summary, 1 mL magnetic beads were concentrated on the side wall of 1 mL tube by magnet, followed by removing the supernatant and resuspending the magnetic beads in 15 mM 2-(N-morpholino)ethanesulfonic acid (MES) buffer pH 6.0. Next, 100 μL of (1-ethyl-3-(3-dimethylaminopropyl)carbodiimide hydrochloride) EDC was added and the magnetic beads were incubated on a roller for 30 minutes. After that, the magnetic beads were washed with a 15 mM MES buffer. Subsequently, magnetic beads were incubated with 400 μg of the desired antibodies overnight on a roller (antibodies were selected using exemplary procedure 3, described below). The next day, magnetic beads were washed three times with 0.1% Tween-20 in phosphate-buffered saline (PBS). The magnetic beads were resuspended in 0.1% BSA dissolved in PBS.

The antibodies used were selected using exemplary procedure 3: Two types of antibodies for Shiga-Toxin-1 were examined based on availability. (anti-Shiga-Toxin-1, Rabbit IgG, referred to as R) was raised against a Stx1a recombinant toxoid, stx1a E167Q, while the second different antibody (anti-Shiga-Toxin, (camelid antibody, VHH, referred to as Vh) is a recombinantly expressed VHH heterotetramer that contains two VHH domains that recognize Stx1. An enzyme-linked immunosorbent assay (ELISA) plate was incubated with the capture antibodies for 16 hours at 4° C. (capture antibody concentration 2.5 μg/mL). Afterward, the plate was incubated for half an hour at 30° C. with mild shaking, followed by three-time washing with 300 μL of Tween-20(0.05 % in PBS). To detect the antigen of interest, 100 μL of interested antigen was incubated for 2 hours at 30° C. with mild shaking. Unbound antigen was washed with 300 μL of Tween-20(0.05 % in PBS) three times. Subsequently, detection antibody-coated AgNPs (50 μL) were added and incubated for two hours at 30° C., followed by washing. To dissolve the AgNPs, 40 μL of HNO₃ was added to each well for 10 minutes. Finally, 100 μL of electrolyte (0.1 M HNO₃+0.1 M of NaNO₃) was added to each well, and 60 μL of that solution was used for electrochemical detection. The experiment was repeated using different combinations of antibodies.

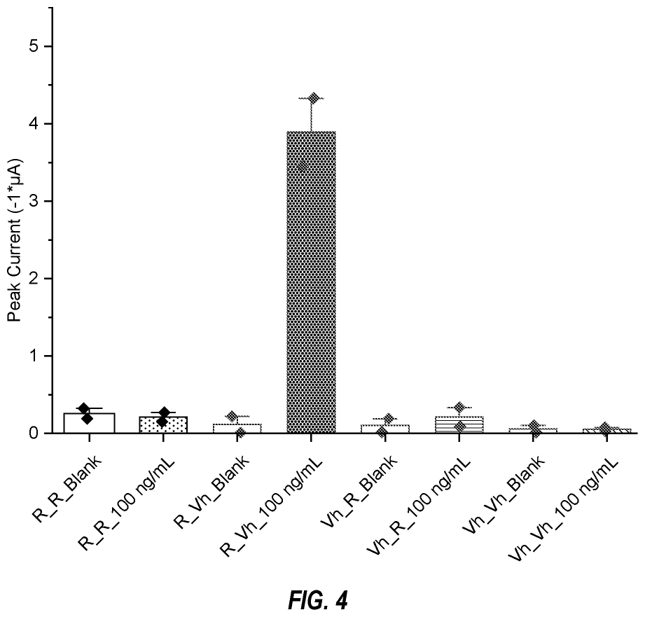

Results from exemplary procedure 3 are illustrated in FIG. 4. As shown in FIG. 4, R_R (the first letter represents the capture antibody, and the second letter represents the detection antibody) has a higher signal than the Vh_Vh pair. However, the R_R pair also increased the blank signal. That means both antibodies bind to the same binding sites of Shiga-Toxin 1. While the Vh_R pair has some signal difference between blank and 100 ng/ml samples, the difference is statistically insignificant. This suggests that Vh also binds to the binding site of the R antibody. By examining the R_Vh pair, it is apparent that the R antibodies bind to different locations on the toxin, which also does not sterically hinder or block the Vh antibody binding sites. Accordingly, using an R_Vh pair represents a particularly advantageous embodiment.

An SPCE electrode may be modified and characterized (e.g., with reference to FIG. 2 and the corresponding description) using exemplary procedure 4: To convert the Ag/AgCl reference electrode's outer surface (surface exposed to liquid) from Ag to AgCl, 1.5 μL of bleach (e.g., sodium hypochlorite bleach) was dripped onto the reference electrode of the SPCEs and the SPCEs were kept in a humidity chamber for 30 minutes. Following that, SPCEs were washed with 10 mL of DI water. Cyclic voltammetry was performed with 1 mM ferro/ferri cyanide in 1 M KCl (50 μL) from −0.1 V to 0.9 V with 10 mV/sec scan rate and 10 mV step size.

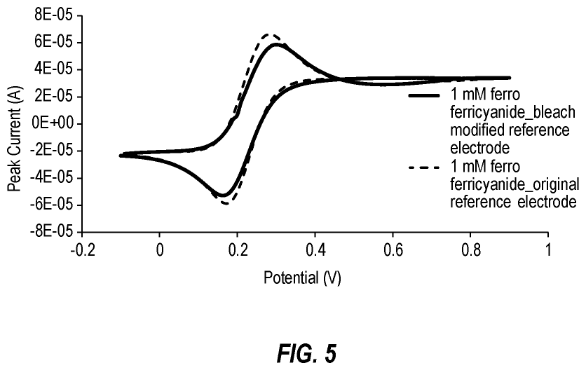

FIG. 5 illustrates the results of exemplary procedure 4. Specifically, the cyclic voltammogram (CV) illustrated in FIG. 5 indicates that SPCEs comprising a reference electrode modified with bleach have substantially the same potential for ferro/ferricyanide (Based on peak position in cyclic voltammetry) as a SPCE comprising a reference electrode without modification. This suggests that the AgNPs' electrochemical peak would appear at substantially the same location and does not need any changes in protocol to deposit or strip silver ions and metal, respectively.

The detection limits of the disclosed methods directed to detecting Shiga toxin-1 concentration (e.g., with reference to FIG. 3 and the corresponding description) were determined using exemplary procedure 5: R-antibody modified 1 μm diameter paramagnetic particles (15 μL) along with antigen (500 μL) and Vh-antibody modified 20 nm AgNPs (50 μL) were incubated for 1 hour on a roller. Next, an assay was washed by replacing the supernatant with 0.05% Tween in PBS using immunomagnetic separation (IMS). Subsequently, an assay was washed three times with 0.05% Tween in PBS. Finally, 40 μL of HNO₃ was incubated in a centrifuge tube for 10 minutes to dissolve the silver nanoparticles, followed by the addition of 100 μL of electrolyte (0.1 M HNO₃+0.1 M of NaNO₃). Part of this solution (60 μL) was used for electrochemical analysis as described below with reference to exemplary procedure 6.

Dissolved AgNPs may be detected and quantified using exemplary procedure 6: AgNPs dissolved in 50% HNO₃ were detected by anodic stripping voltammetry (ASV). Dissolved AgNPs were first deposited on a working electrode by applying −0.5 V for 10 minutes. After depositing silver onto electrodes, it was stripped from the electrode by a square wave voltammetry scan from −0.5 V to 0.5 V (50 mV pulse size, 20 Hz frequency). A peak between 0 to 0.2 V was measured and correlated to Ag concentration.

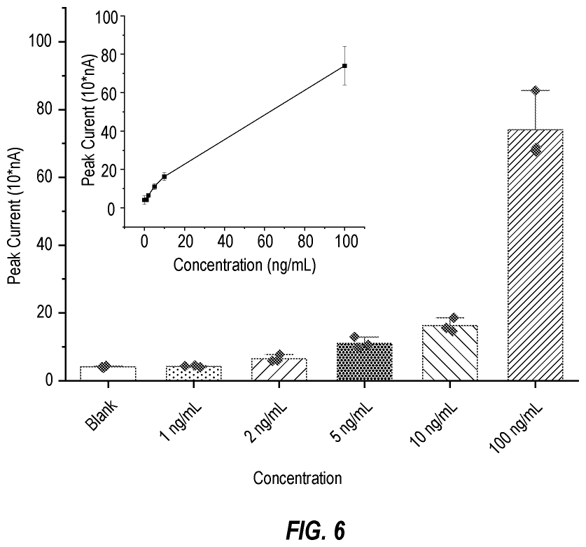

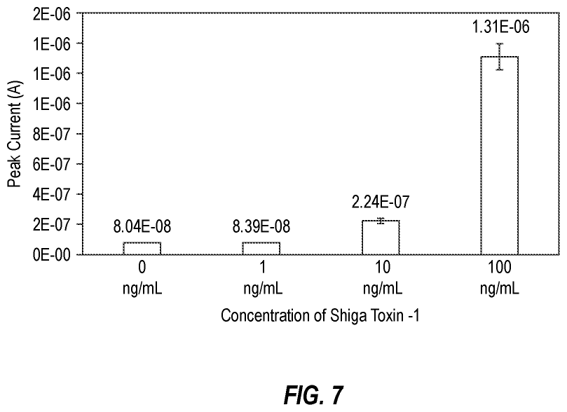

The results of exemplary procedures 5 and 6 are illustrated in FIGS. 6 and 7. FIG. 6 illustrates a bar graph representing the detected peak current for various Shiga toxin-1 concentrations and the inset graph illustrates Shiga-toxin-1 detection in PBS (as described in exemplary procedures 5 and 6). As is apparent, good linearity of the signal is provided (see inset provided in FIG. 6). Furthermore, a student-t test was run on blank to 10 ng/ml samples. Based on this statistical analysis, p-values between blank-1 ng/ml, blank-2 ng/ml, blank-5 ng/ml, blank-10 ng/ml, and 5 ng/ml-10 ng/ml are calculated as 0.90, 0.043, <0.001 ,<0.001 and <0.001 respectively. These p-values suggest that when using AgNPs, 2 ng/ml Shiga toxin-1 detection is possible. This detection limit is within the same order as the manufacturer's Sandwich ELISA's limit of detection. Additionally, signals for 2 ng/ml, 5 ng/mL, and 10 ng/ml are distinguishable from each other. Another set of data for Shiga Toxin-1, which ran on a different day, is illustrated in FIG. 7.

A test to characterize the properties of a comparative electrochemical biosensor using AuNPs was performed using exemplary procedure 7: R-antibody modified 1 μm diameter paramagnetic particles (15 μL) along with antigen (500 μL) and Vh-antibody modified 20 nm AuNP (50 μL) were incubated for 1 hour on a roller. Next, IMS was used to wash an assay three times with 0.05% Tween in PBS. Subsequently, an assay was washed three times with 0.05% Tween in PBS. Finally, an assay was resuspended in 0.1 M HCl in PBS. Part of this solution (60 μL) was used for electrochemical analysis, as described by D. Patel et al., “Sensors and Actuators: B. Chemical Design of a hydrodynamic cavitation system for the extraction and detection of Escherichia coli (O157:H7) from ground beef,” Sensors Actuators B. Chem., vol. 369, no. May, p. 132370, 2022, herein incorporated by reference in its entirety.

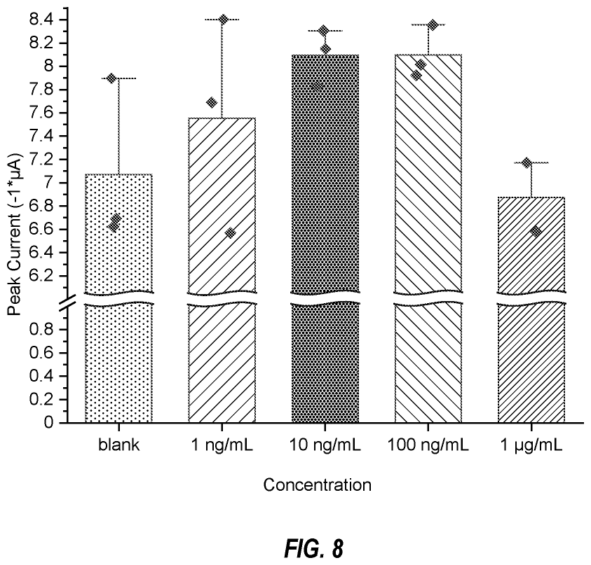

The results of exemplary procedure 7 are illustrated in FIG. 8, which shows a bar graph representing the detected peak current for various Shiga toxin-1 concentrations using an AuNP based electrochemical biosensor. A high standard deviation in the electrochemical signals was observed as shown in FIG. 8. Hence, a one-way analysis of variance (ANOVA) followed by an order difference report was generated using a student t-test. Based on this statistical analysis, p-values between the various experimental pairs blank-1 ng/ml, blank-10 ng/ml, blank-100 ng/ml, and 10 ng/ml-100 ng/ml are calculated as 0.36, 0. 0.072, 0.073 and 0.99 respectively. Therefore, results indicate that Shiga toxin-1 at concentrations of 10 ng/ml and 100 ng/ml are detectable but are not distinguishable from one another. It is further problematic that as shown, the peak current for the blank is substantially similar to the 1 μg/mL toxin concentration.

The results illustrated in FIGS. 6-8 indicate that AgNP-based detection is significantly and statistically better than AuNP-based detection in terms of the limit of detection. Such results are surprising and unexpected. The AuNP-based signal has more variability due to variations in electrochemical dissolution. In contrast, AgNP has more consistent chemical dissolution leading to less variation and better linear trends for graphs of signal vs. concentration. It is likely that further dilution buffer exploration could even further improve the detection limit using the same pair of antibodies and AgNPs (e.g., to detect a toxin concentration of 1 ng/ml or even less).

The embodiments described herein enable the use of a reliable and repeatable method for detecting Shiga toxin-1 despite using low-cost SPCEs. Such methods achieve the same order detection (2 ng/ml) of Shiga toxin as sandwich-ELISA within 3 hours, as described in the certificate of analysis of Shiga toxin antibodies. Due to readily available portable batteries and portable potentiostats, translating this method into a point-of-use diagnostic process is straightforward. In some embodiments, the disclosed methods can be automated by using an electrochemical flow cell.

Without departing from the spirit and scope of this invention, one of ordinary skill can make various modifications to the invention to adapt it to various usages and conditions. As such, these changes and modifications are properly, equitably, and intended to be, within the full range of equivalence of the following claims.

What is claimed is:

1. An electrochemical biosensor comprising:
silver nanoparticles (AgNPs) including antibodies attached thereto; and
a screen printed carbon electrode (SPCE) or other electrode comprising a reference electrode, the reference electrode comprising an outer surface,
wherein the outer surface comprises a silver coating and wherein the outer surface has been passivated.

2. The electrochemical biosensor of claim 1, wherein the outer surface of the reference electrode is passivated by treatment with bleach.

3. The electrochemical biosensor of claim 2, wherein silver chloride is produced on the outer surface.

4. The electrochemical biosensor of claim 2, wherein the outer surface of the reference electrode is substantially free of Ag⁺ ions.

5. The electrochemical biosensor of claim 2, wherein the outer surface of the reference electrode does not significantly contribute to a background signal.

6. The electrochemical biosensor of claim 1, further comprising magnetic nanparticles (MNP) including antibodies attached thereto.

7. The electrochemical biosensor of claim 6, wherein the antibodies attached to the AgNPs comprise Vh-antibodies and the antibodies attached to the MNPs comprise R-antibodies.

8. The electrochemical biosensor of claim 1, wherein the biosensor detects Shiga toxin.

9. The electrochemical biosensor of claim 6, wherein the biosensor detects Shiga toxin at a threshold detection concentration of 2 ng/ml or less.

10. An electrochemical biosensor comprising:
silver nanoparticles (AgNPs) including antibodies attached thereto; and
a screen printed carbon electrode (SPCE) comprising a reference electrode, the reference electrode comprising an outer surface,
wherein the outer surface is passivated with bleach or comprises a platinum coating.

11. The electrochemical biosensor of claim 10, wherein the electrochemical biosensor detects Shiga toxin.

12. An electrochemical detection method, the method comprising:
providing an electrochemical biosensor, the electrochemical biosensor comprising:
silver nanoparticles (AgNPs) including first antibodies attached thereto, and
a screen printed carbon electrode (SPCE) or other electrode comprising a reference electrode, the reference electrode comprising an outer surface,
wherein the outer surface comprises a platinum coating or other passivation coating;
binding the AgNPs to an antigen; and
using the SPCE or other electrode to detect the AgNPs and determine a detected concentration of the antigen.

13. The electrochemical detection method of claim 12, wherein the outer surface of the reference electrode is passivated by treatment with bleach.

14. The electrochemical detection method of claim 13, wherein silver chloride is produced on the outer surface.

15. The electrochemical detection method of claim 13, wherein the outer surface of the reference electrode is substantially free of Ag⁺ ions.

16. The electrochemical detection method of claim 12, wherein the detection method detects Shiga toxin.

17. The electrochemical detection method of claim 16, wherein the method detects Shiga toxin at a threshold concentration of 2 ng/ml or less.

18. The electrochemical detection method of claim 12, wherein using the SPCE to detect the AgNPs comprises dissolving the AgNPs in a buffer comprising nitric acid.

19. The electrochemical detection method of claim 12, wherein the method determines a detected concentration of Shiga toxin or another antigen in less than 3 hours.

20. The electrochemical detection method of claim 12, wherein the antibodies comprise R-antibodies.
